(19) United States
(12) Patent Application Publication
(10) Pub. No.: US 2004/0121373 A1
(43) Pub. Date: Jun. 24, 2004

(54) Fragmentation of DNA

(76) Inventors: Ernest J. Friedlander, San Francisco, CA (US); Lilley Leong, Richmond, CA (US)

Correspondence Address: Finnegan, Henderson, Farabow,, Garrett & Dunner, L.L.P., 1300 I Street, N.W., Washington, DC, 20005-3315, US

(21) Appl. No.: 10/665,916

(22) Filed: Sep. 18, 2003

Related U.S. Application Data
(60) Provisional application No. 60/412,480, filed on Sep. 19, 2002.

(51) Int. Cl. C12Q 1/68; C07H 21/04

(52) U.S. Cl. 435/6; 536/25.3

(57) ABSTRACT

The invention relates to methods and compositions for the fragmentation of DNA.

### PRIORITY DATA

This application is a continuation of U.S. patent application Ser. No. 60/412,480, filed Sep. 19, 2002. application Ser. No. 60/412,480 is incorporated herein in its entirety for any purpose.

### FIELD OF THE INVENTION

The invention relates to methods and compositions for the fragmentation of DNA.

### BACKGROUND

Fragmentation of nucleic acid is often desirable in nucleic acid analysis. The analysis of nucleic acid sequences in a sample containing one or more target sequences is commonly practiced. For example, the detection of genetic or heritable diseases, such as phenylketonuria, routinely includes screening genomic DNA for the presence or absence of diagnostic nucleic acid sequence. Also, detecting the presence or absence of nucleic acid sequences is often used in forensic science, paternity testing, genetic counseling, and organ transplantation.

### SUMMARY OF THE INVENTION

In certain embodiments, methods for fragmenting DNA are provided. In certain embodiments, these methods comprise incubating the DNA above 90° C. In certain embodiments, the DNA is incubated in a composition that is substantially free of nuclease. In certain embodiments, the incubation occurs in a thermal cycling apparatus.

In certain embodiments, methods for fragmenting genomic DNA are provided. In certain embodiments, these methods comprise incubating the genomic DNA above 90° C.

### BRIEF DESCRIPTION OF THE FIGURES

FIG. 1A shows a gel electrophoresis of certain mechanically fragmented genomic DNA.

FIG. 1B shows a gel electrophoresis of intact (I) and sheared (S) DNA that was boiled for different lengths of time, and DNAse I treated (D) DNA, as discussed in Example 1.

FIG. 2 shows the effects of genomic DNA concentration and boiling duration on two different sources of genomic DNA, as discussed in Example 1.

FIG. 3 shows the effects of boiling for different lengths of time on 4 different genomic DNA sources, as discussed in Example 1.

FIG. 4 compares the assay of intact gDNA with boiled DNA as discussed in Example 1.

FIG. 5A shows a gel electrophoresis of OLA/PCR products generated from different fragmented genomic DNA sources, as discussed in Example 1.

FIG. 5B shows capillary electrophoresis of OLA/PCR products generated from different fragmented genomic DNA sources, as discussed in Example 1.

FIG. 6 shows the results of hybridization of PE-27 planar arrays to OLA/PCR products generated from genomic DNA fragmented by DNAse I treatment, boiling for 15 minutes, and boiling for 30 minutes, as discussed in Example 1.

FIG. 7 shows the results of hybridization of PE-27 planar arrays to OLA/PCR products generated from intact genomic DNA, gDNA fragmented by boiling 15 minutes, and gDNA fragmented by boiling 60 minutes, as discussed in Example 1.

### DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Definitions

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-′-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures:

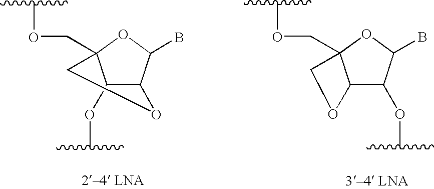

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

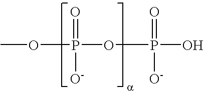

where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

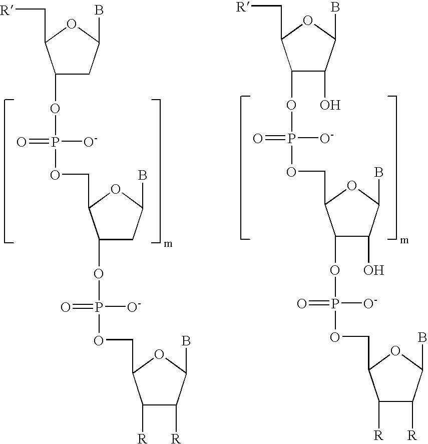

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or

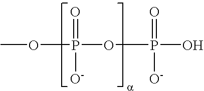

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The term “fluorescent indicator” refers to any molecule or group of molecules that emit a fluorescent signal at an intensity relative to the amount of a nucleic acid present. Non-limiting examples of such fluorescent indicators include fluorescent dyes and 5′ nuclease probes.

“Fluorescent dyes” refer to those molecules that fluoresce and bind to nucleic acid. Certain exemplary fluorescent dyes may bind preferentially, or with a higher affinity, to double-stranded nucleic acid than to single-stranded nucleic acid. Examples of such fluorescent dyes that exhibit preferential binding to double-stranded nucleic acid include, but are not limited to, minor groove-binding dyes and intercalating dyes, such as SybrGreen™ (Sigma, St. Louis, Mo.) and PicoGreen™ (Molecular Probes, Eugene, Oreg.).

The term “fragmentation” refers to the breaking of nucleic acid molecules into smaller nucleic acid fragments. In certain embodiments, the size of the fragments generated during fragmentation can be controlled such that the size of fragments is distributed about a certain predetermined nucleic acid length. Several methods of fragmentation are available, including, but not limited to, boiling, heating, and mechanical shearing. One method of fragmentation is sonication or the use of ultrasound, which is distinguishable from conventional heating by the use of ultrasonic waves that disrupt and fragment nucleic acid molecules.

In certain embodiments, “fragmented” nucleic acid molecules are those where at least 50% of the nucleic acid molecules subjected to fragmentation have been broken into smaller nucleic acid fragments. In certain embodiments, “fragmented” nucleic acid molecules are those where at least 20% of the nucleic acid molecules subjected to fragmentation have been broken into smaller nucleic acid fragments.

The terms “quantitate” and “quantitation” refer to measuring the quantity of an analyte or a total amount of a nucleic acid in a sample. In certain embodiments, the total amount of nucleic acid in a sample may be quantitated. In certain embodiments, the total amount of a specific nucleic acid sequence may be quantitated.

The term “thermal cycling apparatus” refers to any apparatus that is designed for the incubation of nucleic acid samples at specifically programmed temperatures and can change temperatures in a programmed manner. A non-limiting example of such an apparatus is a device designed for the thermal cycling of PCR reactions.

The term “a composition that is substantially free of nuclease” refers to a composition in which there is insufficient nuclease to effect substantial fragmentation of the DNA.

The term “substantial fragmentation of the DNA” refers to the fragmentation of more than 30% of DNA molecules in a sample into smaller nucleic acid fragments. In certain embodiments, “substantial fragmentation of DNA” refers to the fragmentation of at least 50% of the DNA molecules in a sample into smaller nucleic acid fragments. In certain embodiments, “substantial fragmentation of DNA” refers to the fragmentation of at least 80% of the DNA molecules in a sample into smaller nucleic acid fragments. In certain embodiments, “substantial fragmentation of DNA” refers to the fragmentation of at least 90% of the DNA molecules in a sample into smaller nucleic acid fragments. In certain embodiments, “substantial fragmentation of DNA” refers to the fragmentation of at least 95% of the DNA molecules in a sample into smaller nucleic acid fragments.

Certain Exemplary Embodiments

Fragmentation of nucleic acids comprises breaking nucleic acid molecules into smaller fragments. Fragmentation of nucleic acid may be desirable to optimize the size of nucleic acid molecules for certain reactions. According to certain embodiments, fragmented nucleic acids are used when performing certain assays. For example, in certain embodiments, fragmentation of genomic DNA may allow more efficient hybridization of genomic DNA to nucleic acid probes than nonfragmented genomic DNA.

Certain methods of fragmentation of nucleic acid employ enzymatic and mechanical methods. A non-limiting example of an enzymatic method includes incubation with DNAse I. DNAse I, diluted to sufficiently low concentrations to prevent total degradation of the DNA, will break large double-stranded DNA molecules into smaller fragments of DNA.

Mechanical methods of fragmentation include, but are not limited to, passing nucleic acids, such as genomic DNA, through small tubes, holes, filters, or syringes.

According to certain embodiments of the invention, methods of fragmentation comprise incubation of nucleic acid at elevated temperatures. In certain embodiments, the temperatures for incubation range from 80° C. to 100° C. In certain embodiments, the temperature for incubation ranges from 90° C. to 99° C. In certain embodiments, the temperature for incubation is 95° C. Several methods are available for incubating nucleic acids at elevated temperatures. These methods include, but are not limited to, using heating blocks, ovens, water baths, incubators, or a thermal cycling apparatus. In certain embodiments of the invention, the incubation occurs in a thermal cycling apparatus. In certain embodiments of the invention, the DNA is incubated in a composition that is substantially free of nuclease.

In certain embodiments, methods for fragmenting nucleic acid are provided. In certain embodiments, these methods comprise incubating the nucleic acid above 90° C. In certain embodiments the incubation occurs in a solution comprising 10 mM Tris and 1 mM EDTA. In certain embodiments, the solution is pH 8. In certain embodiments, the pH is between 7 and 9. In certain embodiments, the pH is between 6 and 10.

In certain embodiments, the incubation lasts between 5 and 60 minutes. In certain embodiments, the incubation lasts between 15 and 30 minutes. Some experiments have shown that very high concentrations of gDNA (mg quantities) yield fragments in the 100-800 bp range after hundreds of cycles. In such instances, the incubation can be as short as 1-2 seconds for each cycle, and the total incubation can be less than 5 minutes.

One may use DNA fragmented according to certain embodiments of the invention for various procedures. Certain exemplary procedures include, but are not limited to, ligation assays, nucleic acid amplification assays such as PCR, and ligation and amplification assays. Exemplary ligation and/or amplification assays include, but are not limited to, those discussed in Genomics, 29:152-162 (1995); J. Mol. Biol., 292:251-262 (1999); Nucleic Acids Res., 27:1810-1818 (1999). Exemplary ligation and amplification assays include, but are not limited to, those discussed in U.S. Pat. No. 6,027,889, PCT Published Patent Application No. WO 01/92579, and U.S. patent application Ser. Nos. 09/584,905 and 10/011,993.

### EXAMPLE I

Test gDNA was used to evaluate several fragmentation methods. In this work, two different types of test gDNA was used. Intact gDNA was used in certain work. Also, to mimic poor quality gDNA in which the gDNA may not be fully intact, gDNA was sheared to a limited extent to create test sheared gDNA for certain work. For this example, the cell-line gDNA was obtained from Coriell (Camden, N.J.), while the blood gDNA was extracted from blood purchased from Stanford Medical Center using the QiaAmp DNA blood midi kit (QlAgen Part No. 51192). Test sheared gDNA was generated by passing intact gDNA sequentially through 18-gauge, 22-gauge, and 25-gauge needles in 10 mM Tris-1 mM Na₂EDTA, pH 7.4.

The test gDNA was fragmented by mechanical fragmentation, boiling, or DNase I-digestion. Mechanical fragmentation of gDNA was achieved by rapidly pipetting intact or test sheared gDNA through 400 μm capillaries to simulate the mechanical fragmentation of gDNA described by Oefner et al. Nucleic Acids Res., 24: 3879-3881, (1996)).

Boiling was performed by incubating intact or test sheared gDNA (33.5-360 ng/μL) in 1×TE (pH 7.4 or pH 8.0) at 99° C. for different durations.

DNAse I fragmentation was performed by preparing a chilled reaction mixture containing gDNA (˜100 ng/μL final concentration), 0.01 U/μL (final concentration) DNase I, 50 mM Tris-HCl (pH 7.6) and 10 mM MgCl₂. The cold reaction mixture was transferred to a thermocyler, where the DNase I digestion of DNA and enzyme inactivation were performed. The cycling parameters used were: 25° C., 20 minutes, 99° C., 15 minutes. The DNase I-digested DNA was stored at −20° C. until use.

Comparison of Different Fragmentation Methods

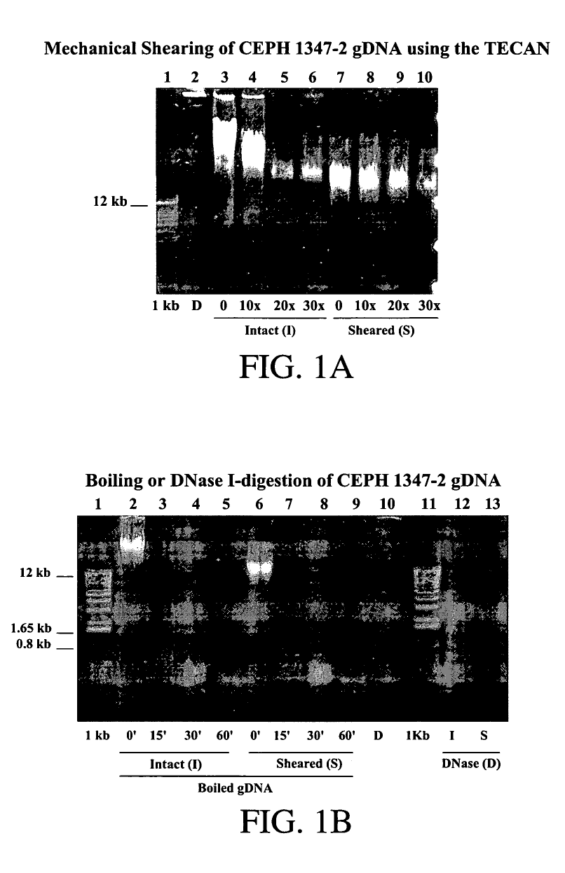

FIG. 1 compares the different methods of fragmenting gDNA in terms of the size of gDNA fragments that were generated. The fragments were detected by agarose gel electrophoresis. FIG. 1A shows that mechanical shearing was not optimal since the fragment sizes that were generated were larger than 12 kb. FIG. 1B demonstrates that boiling and DNase I digestion (D) were more effective since the size of fragments that were generated was less than or equal to 3 kb.

Also, the gDNA was digested with DNase I by two different individuals on different occasions. The individuals used the same protocol discussed above. The results are shown in lane 1 of FIG. 1A, and lanes 10, 12 and 13 of FIG. 1B. The fragment size distribution differed significantly between the two different DNAse I fragmentations, which may have resulted from different individuals carrying out the same protocol. This illustrates that it can be difficult to achieve uniformity of preparation with DNase I treatment.

FIG. 1B also shows that the quality of the starting gDNA (intact gDNA versus test sheared gDNA) had little to no effect on the fragment sizes that were generated when the test gDNA was boiled. Specifically, similar fragment sizes were obtained with the boiling of either intact gDNA or test sheared gDNA.

Also, FIG. 1B shows that the size of the generated fragments decreased with each of the tested increased durations of boiling.

The remainder of the studies in this Example were performed with intact gDNA boiled in 1×TE (pH 8.0). In certain embodiments, TE at other concentrations or pH may be used.

Testing of Concentration of gDNA, Boiling Duration, and Source of gDNA

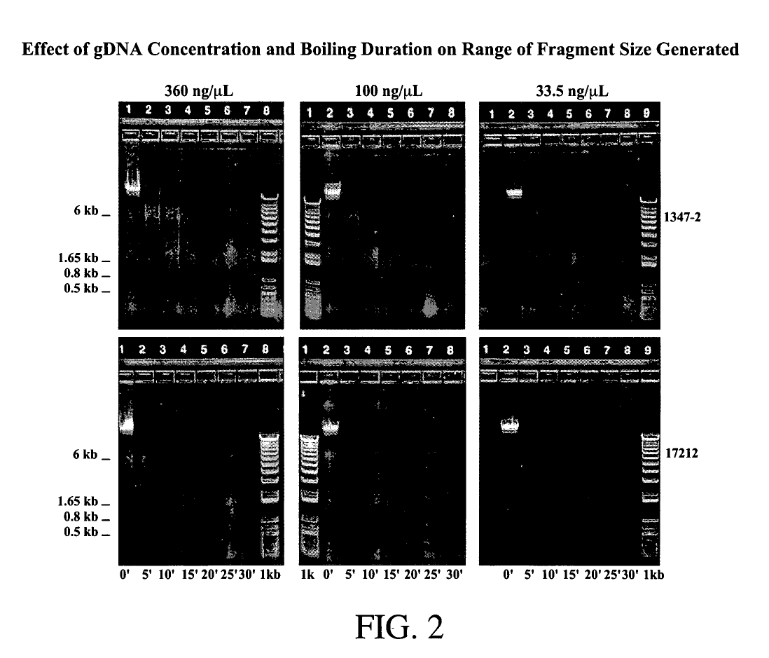

Two different sources of gDNA, CEPH 1347-2 and NA-17212, were each subjected to boiling at three different concentrations. Different concentrations of DNA (33.5 ng/μl, 100 ng/μl, or 360 ng/μl in 1×TE (pH 8)) were prepared for each of the CEPH 1347-2 gDNA and the NA-17212 gDNA. Each of the three concentrations of gDNA for each of the two gDNA sources (six different possibilities) was placed into seven different tubes for analysis as follows: (1) no boiling; (2) boiling for 5 minutes; (3) boiling for 10 minutes; (4) boiling for 15 minutes; (5) boiling for 20 minutes; (6) boiling for 25 minutes; and (7) boiling for 30 minutes. After the desired duration of boiling, each of the six different tubes containing one of the two different sources of gDNA, at one of the three different concentrations, were removed from the thermocycler and immediately placed on ice. Then, 0.5 μg of the gDNA from each tube was placed in a lane and subjected to agarose gel electrophoresis. The results are shown in FIG. 2.

The results showed that the degree of fragmentation achieved by boiling in this work was a function of the initial gDNA concentration and boiling duration (see FIG. 2). Either prolonging the boiling time or decreasing initial gDNA concentration resulted in the generation of smaller fragments between 100-800 bp. In contrast, the volume of gDNA boiled did not appear to have any effect on the size distribution, since preliminary experiments comparing 60 μl and 150 μL of CEPH 1347-2 gDNA appeared to show a similar fragment size distribution when boiled for 15 minutes in 1×TE, pH 8 (data not shown).

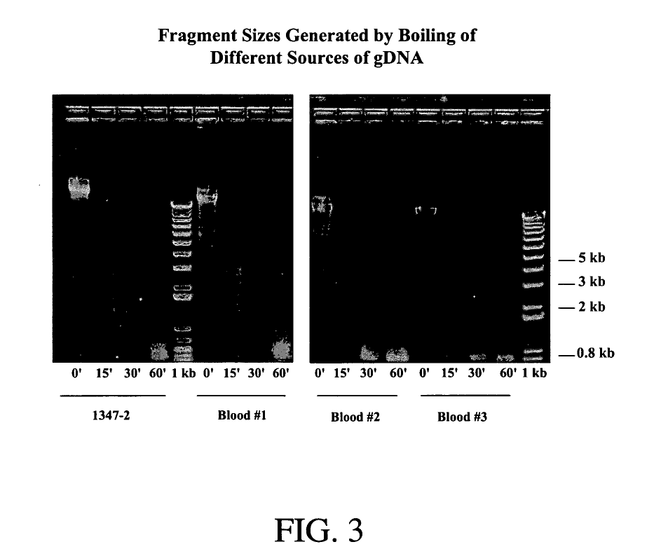

Fragmenting gDNA by boiling was tested with a variety of cells, gDNA from different cell lines (including CEPH 1347-2), and blood cells obtained from 3 different donors. For each sample, gDNA was boiled for 0, 15, 30, and 60 minutes at a concentration of 100 ng/μl in 1×TE, pH 8 (FIG. 3). After boiling, 0.5 μg of each of the samples was loaded onto a lane of 0.8% agarose gel. Examination of the range of fragment sizes generated indicated that similar-sized fragments were obtained for a given duration of boiling, regardless of whether the gDNA was derived from a cell line or from blood cells.

Testing of Fragmented gDNA

The products that were obtained by boiling intact gDNA were assessed in several ways, including by TaqMan® assay for the RNase P, by Oligonucleotide Ligation Assay, Polymerase Chain Reaction (OLA/PCR), and by the hybridization of the generated OLA/PCR products to planar PE-27 arrays, a screen for 40 specific Single Nucleotide Polymorphisms (SNPs).

TaqMan Assay for RNase P

The TaqMan® assay for RNase P (Applied Biosystems, Cat. No. 4316831) was carried out according to the RNAse P TaqMan® kit specifications, using the DNA standard and FAM-labelled probes provided with the kit for RNase P and the TaqMan® Universal PCR Master Mix (Applied Biosystems, Cat. No. 4305719).

The TaqMan® assay for RNase P allowed both the quantitation of the fragmented gDNA and the assessment of the gross integrity of the fragmented gDNA. In a TaqMan® assay for RNase P, the probe binds to the RNase P gene or on copies of the gene on the gDNA. As Taq Polymerase synthesizes additional copies of the gDNA (and the RNase P gene), it cleaves the RNase P probe bound to the RNase P gene. The RNase P probe is designed in such a way that cleavage of the probe results in the generation of a fluorescent signal, which may be detected at each cycle of Taq polymerase amplification of gDNA. Since there is a direct relationship between the starting amount of DNA present and the amount of DNA synthesized by Taq Polymerase in the early stages of PCR, determination of the cycle at which RNase P probe fluorescence is detectable (Ct or threshold cycle) was used to determine the starting gDNA amount.

The RNase P content of gDNA preparations containing small fragments (highly fragmented) would be expected to be drastically reduced compared to that of preparations containing larger fragments. Consistent with these expectations, DNAse I-treated gDNA demonstrated a reduction in the amount of RNase P detected when compared to boiled gDNA (data not shown).

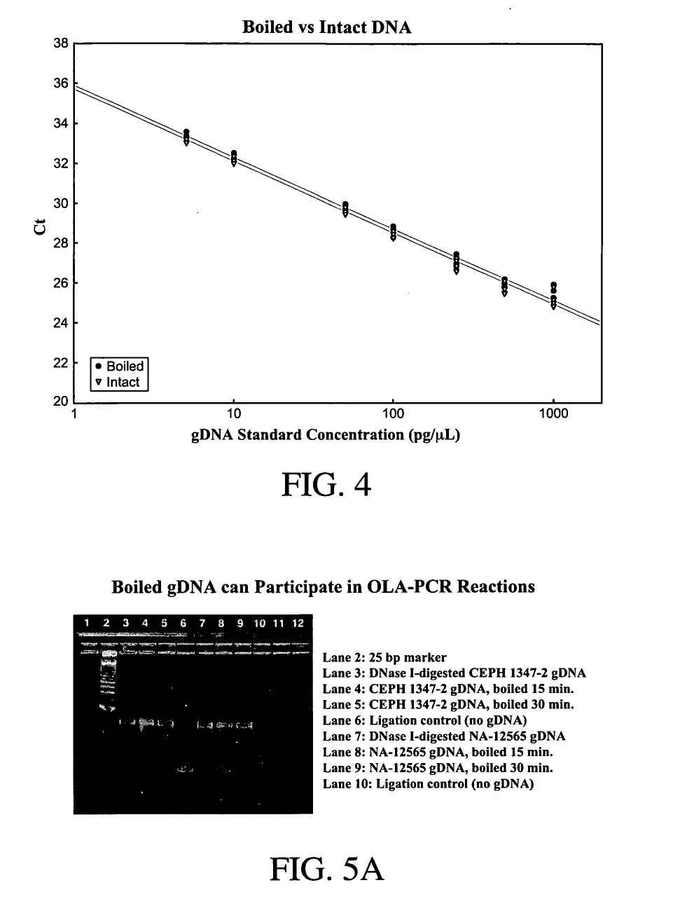

In the concentrations that were tested, gDNA which was unboiled (intact) or boiled for 15 minutes showed very similar RNase P assay results (See Table 1 and FIG. 4). In FIG. 4, the plots of the log (starting DNA concentration) and the Ct value for both boiled and intact DNA are linear and are nearly coincident. The data suggested that in this work there were no profound changes in the integrity of the DNA that was fragmented by boiling relative to the intact DNA.

TABLE 1
Effect of Boiling on RNase P Concentrations of Blood gDNA

| | |
|---|---|
| Intact | Boiled |
| RNase P | RNase P |
| Concentrations | Concentrations |

| Sample | average | stdev | average | stdev |
|---|---|---|---|---|
| 1 | 165.0 | 17.3 | 192.5 | 17.1 |
| 2 | 227.5 | 20.6 | 262.5 | 12.6 |
| 3 | 140.0 | 14.1 | 152.5 | 12.6 |
| 4 | 195.0 | 17.3 | 190.0 | 14.1 |
| 5 | 237.5 | 17.1 | 255.0 | 12.9 |
| 6 | 207.5 | 9.6 | 257.5 | 9.6 |
| 7 | 120.0 | 8.2 | 150.0 | 16.3 |
| 8 | 245.0 | 17.3 | 277.5 | 5.0 |
| 10 | 127.5 | 9.6 | 140.0 | 8.2 |

Probing for Different SNPs by OLA/PCR Assay

OLA/PCR assays were performed with DNA, which were unfragmented (intact), boiled, or DNase I-treated. Also as a measure of nonspecific ligation, DNA was omitted from some reactions. Two sources of gDNA, CEPH 1347-2 and NA 12565, were subjected to boiling for 15 minutes or 30 minutes at starting DNA concentrations of ˜300 ng/μl in 1×TE (pH 8), or to DNAse I digestion, using the procedures discussed above. The fragmented DNA was used for OLA/PCR assays.

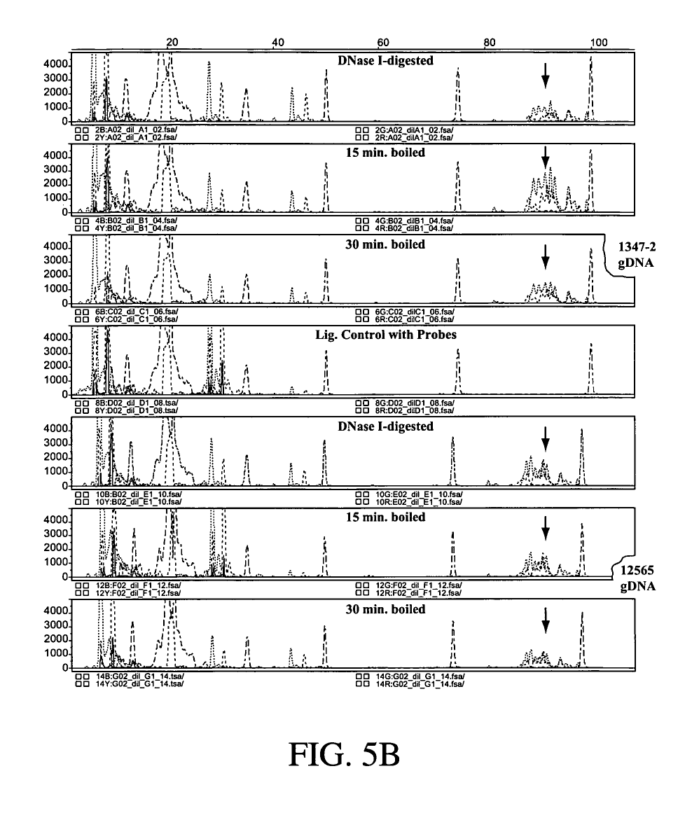

OLA/PCR products were detected by both gel and capillary electrophoresis, although no product was detected in reactions without gDNA (ligation control, FIGS. 5A and 5B).

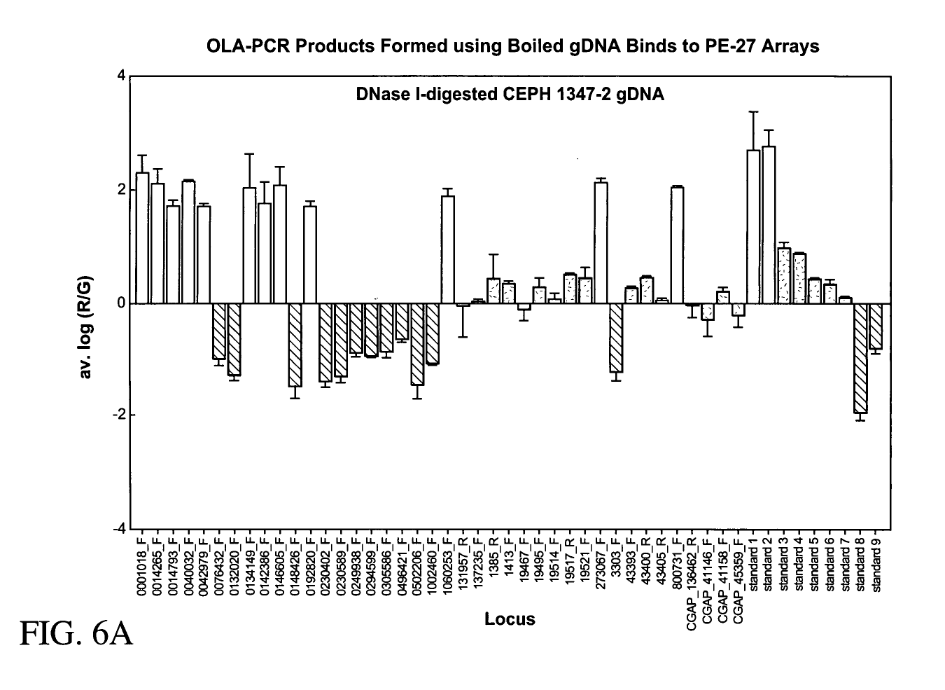

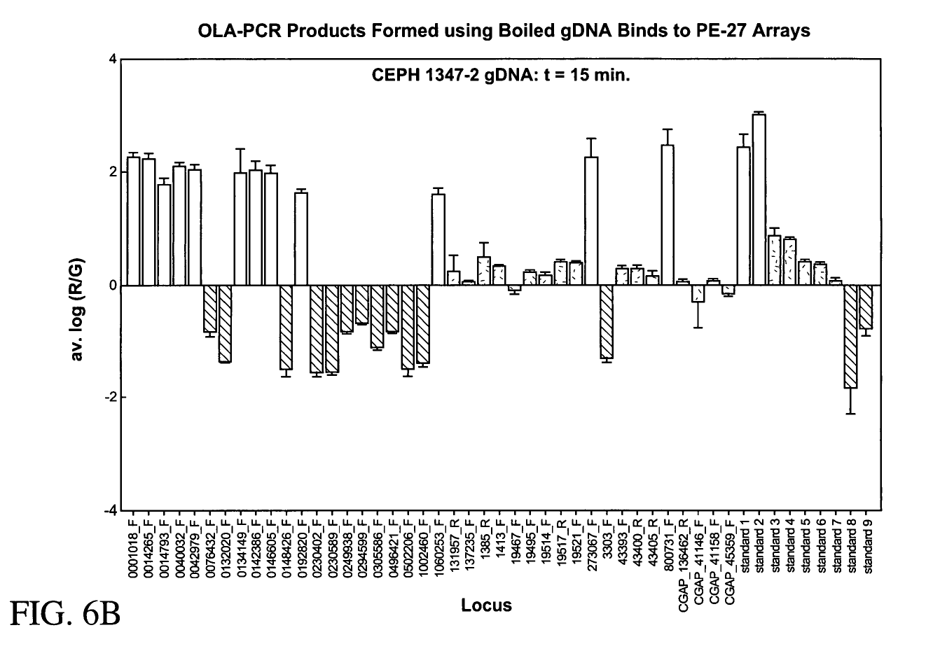

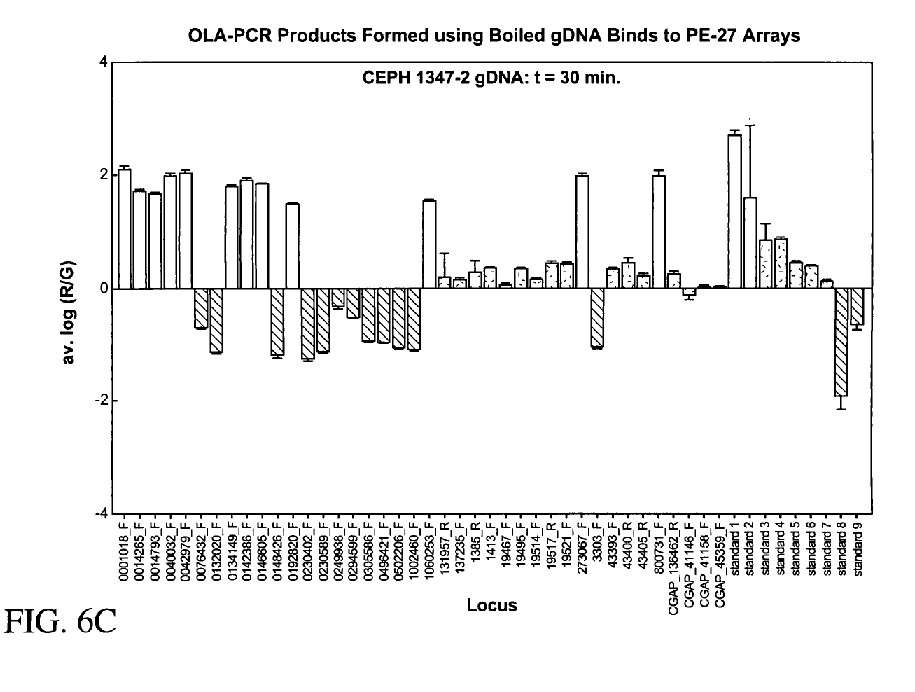

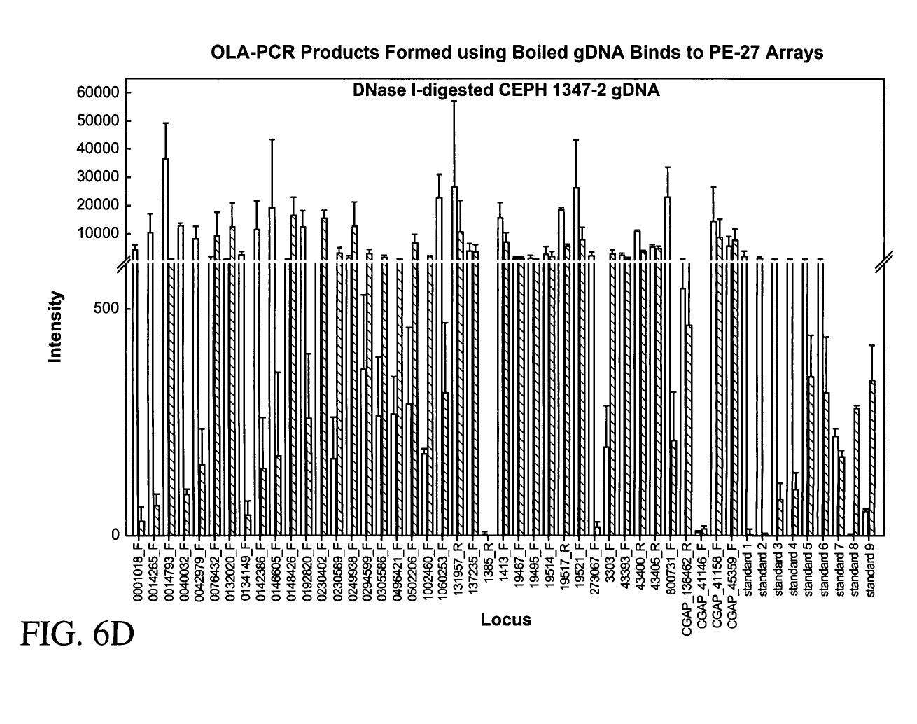

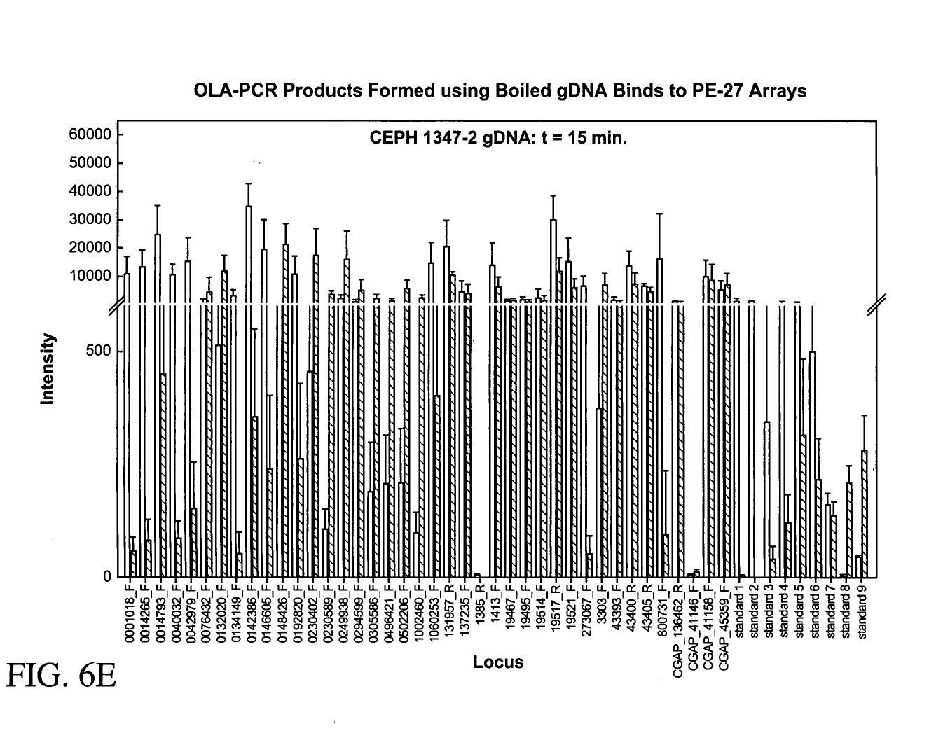

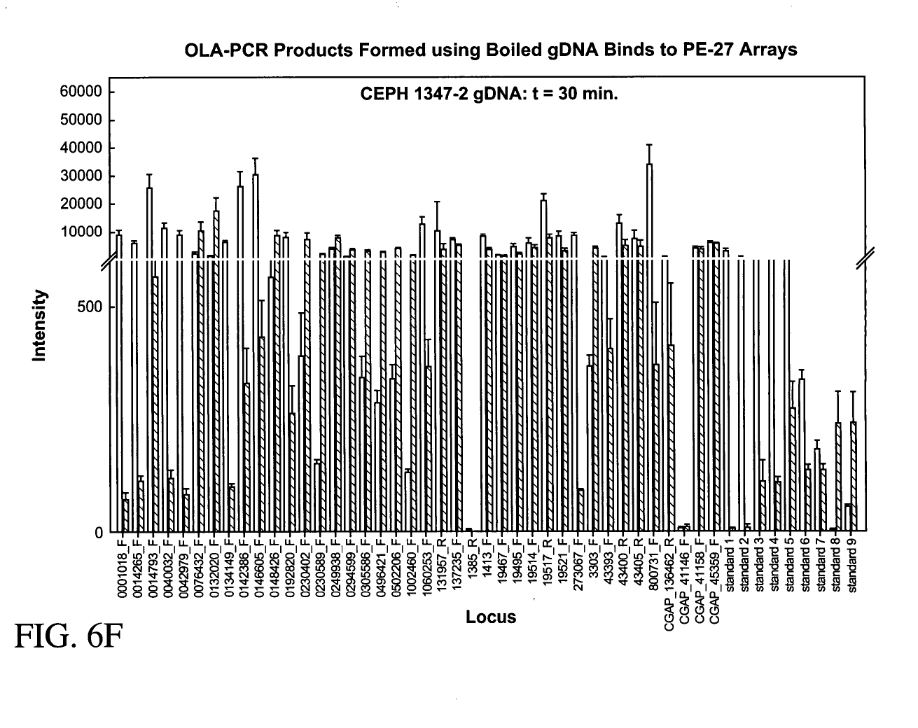

Hybridization to PE-27 arrays allows one to identify the SNP present in the gDNA target, as well as a determination of whether one of two alleles is present or whether both alleles are present. PE-27 arrays are arranged such that the detection of fluorescence at a specific location on the array will indicate the presence of a specific SNP. The fluorescence allows the identification of alleles expressed. Red fluorescence (positive log R/G) or green fluorescence (negative log R/G) indicates homozygosity for either allele, while yellow fluorescence (log R/G ˜1) indicates heterozygosity. The OLA/PCR products that were formed in this work using either DNase I-digested or boiled gDNA appeared to hybridize similarly to PE-27 planar arrays (FIG. 6).

Comparison of Intact and Boiled gDNA in OLA/PCR Assays

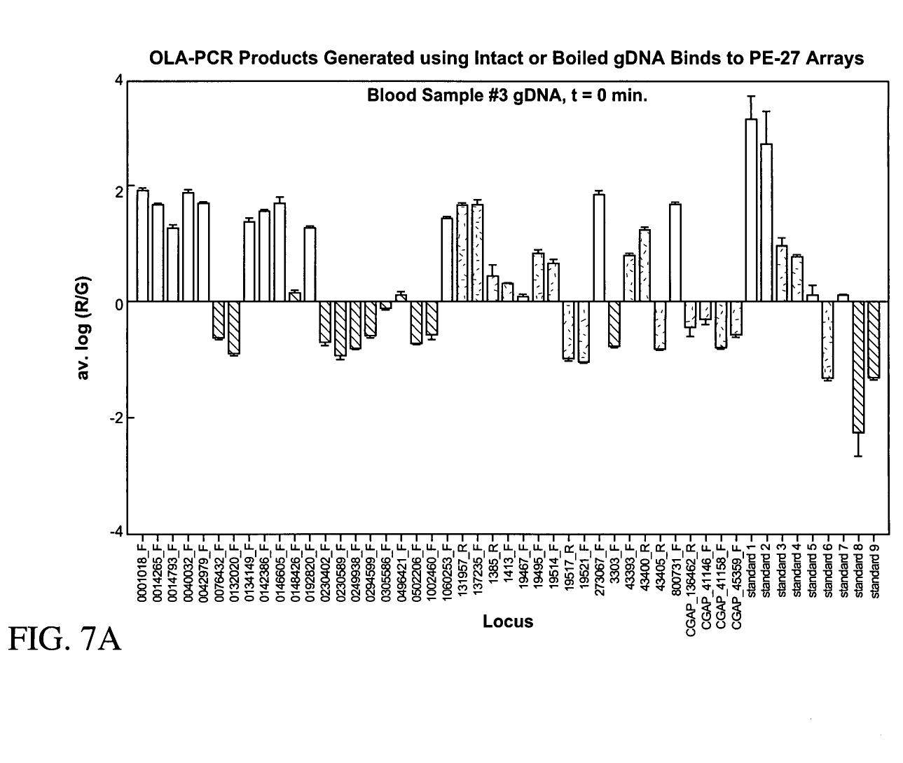

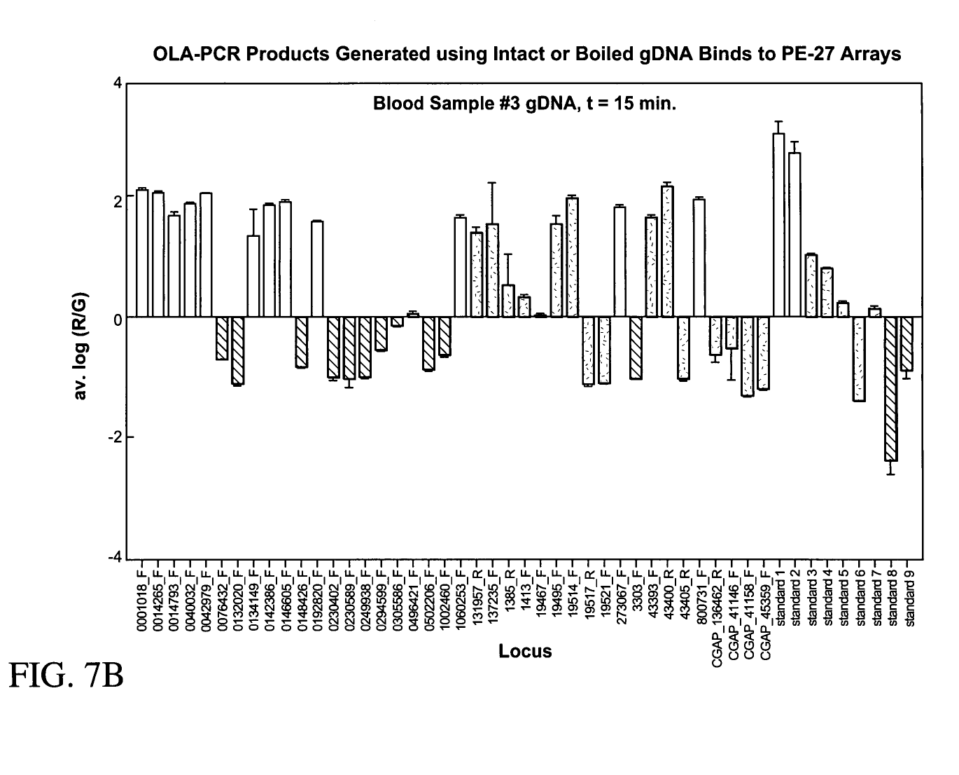

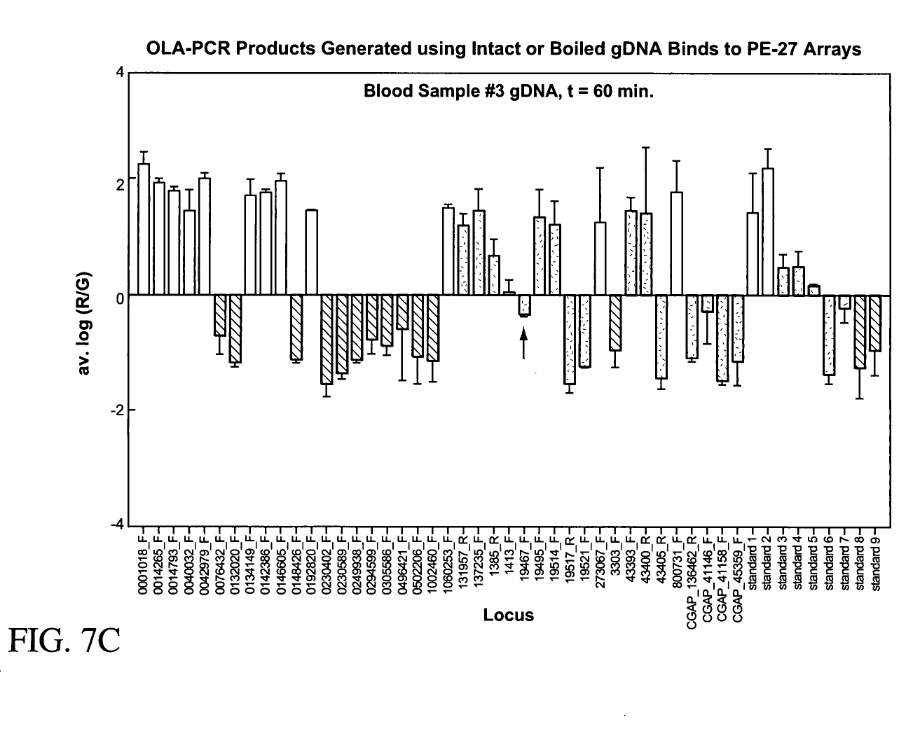

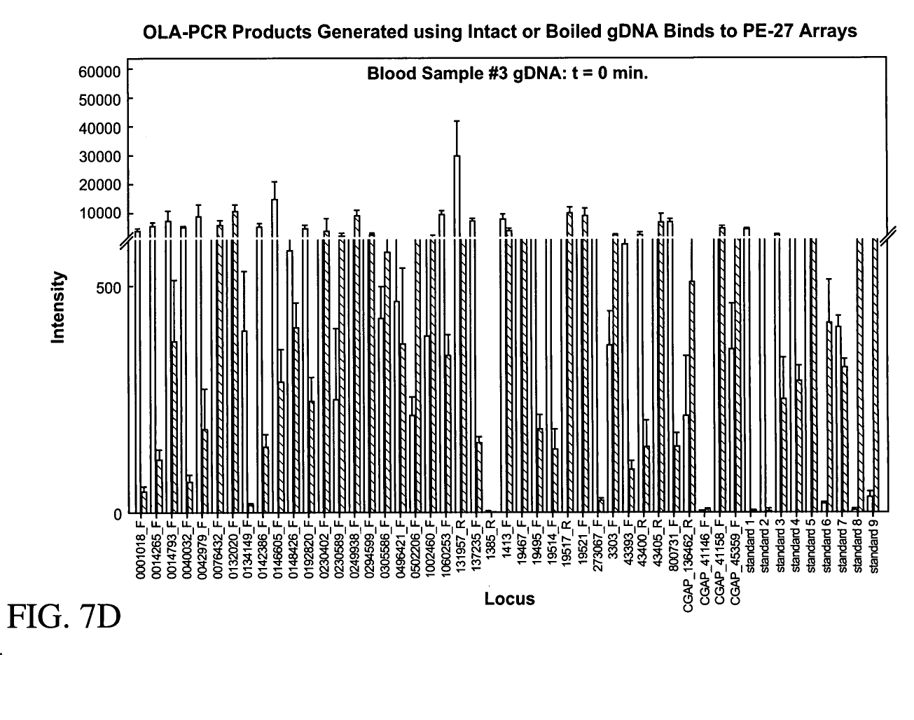

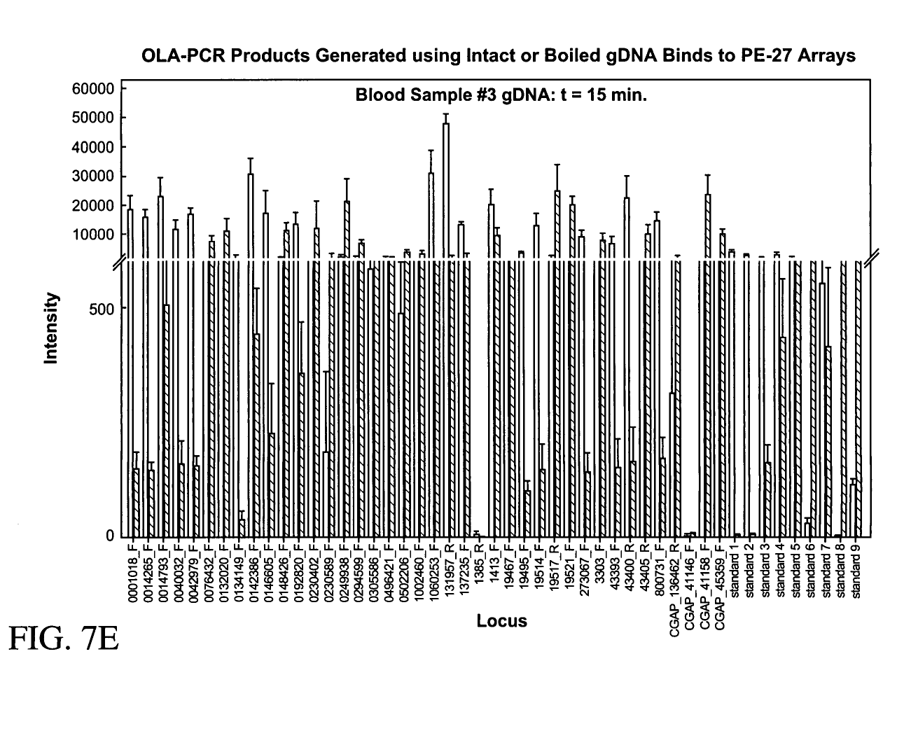

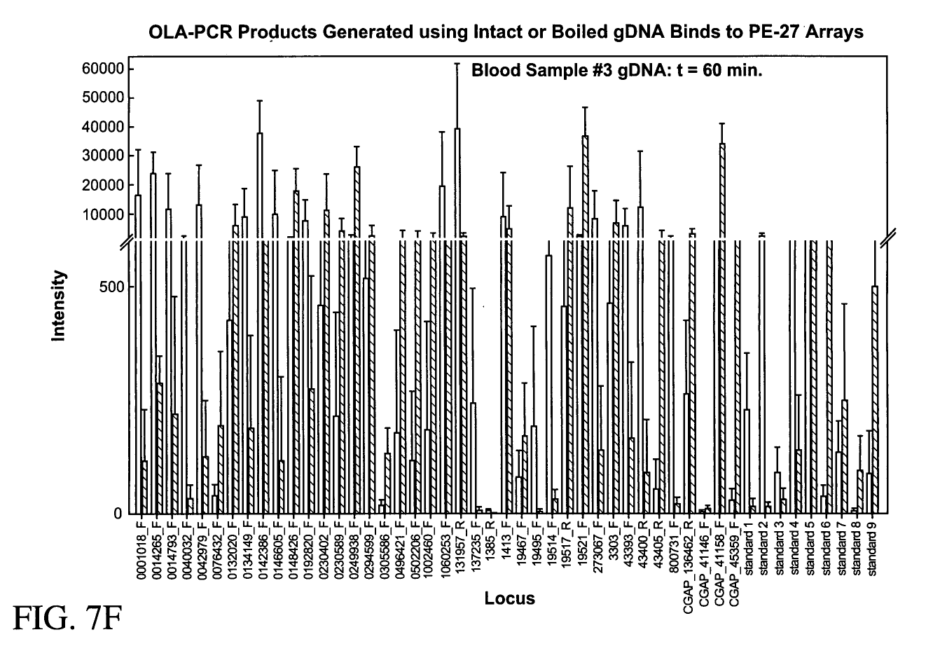

Genomic DNA from blood was boiled in 1×TE (pH 8) at a concentration of 100 ng/μl. One tube of the gDNA was not boiled, one tube of the gDNA was boiled for 15 minutes, and one tube of the gDNA was boiled for 15 minutes (See FIG. 7). The gDNA that was not boiled (0 minutes) did not fragment. Then, the gDNA or fragmented gDNA was subjected to OLA/PCR reactions as described above. The OLA/PCR products obtained from the reactions were analyzed in parallel, either by subjecting to capillary electrophoresis (not shown) or by hybridization to PE-27 planar arrays (FIG. 7). As in FIG. 6, homozygous SNPs are indicated by diagonal or horizontal bars. However, unlike FIG. 6, other SNPs are depicted in gray—these represent SNPs whose genotypes are unknown a priori.

Similar genotype assignments were made, regardless of whether the gDNA was boiled or not (FIG. 7, panels on the left). However, boiling for 60 minutes resulted in much smaller fragments (less than 800 bp), which may adversely affect genotype separation. In the OLA/PCR products generated using gDNA, which was boiled for 60 vs 15 minutes, there was a minor conversion of positive log (R/G) in some heterozygote SNPs to negative log (R/G)—see arrows, increased variance associated with the individual SNP log ratios, and decreased fluorescence intensity associated with some SNPs (FIG. 7, bottom right panel).

What is claimed is:

1. A method of fragmenting DNA comprising incubating the DNA above 90° C. in a composition that is substantially free of nuclease.

2. The method of claim 1, wherein the DNA is in a solution comprising 10 mM Tris and 1 mM EDTA.

3. The method of claim 2, wherein the solution is pH 8.

4. The method of claim 1, wherein the incubation lasts between 5 and 60 minutes.

5. The method of claim 1, wherein the incubation lasts between 15 and 30 minutes.

6. The method of claim 1, wherein the DNA is quantitated after fragmentation.

7. The method of claim 6, wherein the composition comprises a fluorescent indicator.

8. The method of claim 7, wherein the fluorescent indicator is selected from a group comprising a fluorescent dye and a 5′-nuclease probe.

9. A method of determining the presence or absence of a DNA sequence in a sample comprising:
generating a quantity of fragmented DNA comprising incubating the nucleic acid above 90° C. in a thermal cycling apparatus in a composition that is substantially free of nuclease,
quantitating the fragmented DNA, and
performing an oligonucleotide ligation assay, and
determining the presence or absence of the DNA sequence from the oligonucleotide ligation assay.

10. A method of determining the quantity of a DNA sequence in a sample comprising:
generating a quantity of fragmented DNA comprising incubating the nucleic acid above 90° C. in a thermal cycling apparatus in a composition that is substantially free of nuclease,
quantitating the fragmented DNA, and
performing an oligonucleotide ligation assay, and
determining the quantity of the DNA sequence from the oligonucleotide ligation assay.
